SEQUENCE LISTING (16 sequences)

SEQ ID NO: 1 - protein, 107 aa, Mus musculus
SIVMTQTPKFLLVSAGDRVTMTCKASQSVSSDVGWYQQKPGQSPKLLIYSGSNRYSGVPDRFTGSGYGTDFTFTISTVQAEDLAVYFCQQDYYSPWTFGGGTKLEIK

SEQ ID NO: 2 - protein, 107 aa, Artificial Sequence
  Synthesized
DIQMTQSPSSLSASVGDRVTITCKASQSVSSDVGWYQQKPGKAPKLLIYSGSNRYSGVPSRFSGSGSGTDFTLTISSLQPEDFATYYCQQDYYSPWTFGQGTKVEIK

SEQ ID NO: 3 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

SEQ ID NO: 4 - protein, 121 aa, Mus musculus
EVHLQQSGPELVKPGASVKMSCKASGYTFTNYVINWVKQKPGQGLEWIGYNDPYNDVSKYNEKFKGKATLTSDKSSSTAYMELSSLTSEDSAVYYCAKEGGGKYVYAMDSWGQGTSVTVSS

SEQ ID NO: 5 - protein, 121 aa, Artificial Sequence
  Synthesized
EVQLVQSGAEVKKPGSSVKVSCKASGYTFTNYVINWVRQAPGQGLEWIGYNDPYNDVSKYNEKFKGRATITSDKSTSTAYMELSSLRSEDTAVYYCAKEGGGKYVYAMDSWGQGTTVTVSS

SEQ ID NO: 6 - protein, 122 aa, Homo sapiens
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARVGQLGYYYYGMDVWGQGTTVTVSS

SEQ ID NO: 7 - protein, 123 aa, Artificial Sequence [sequence omitted]

SEQ ID NO: 8 - protein, 108 aa, Artificial Sequence
  Synthesized
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKR

SEQ ID NO: 9 - protein, 123 aa, Mus musculus
EIQLVQSGPELKQPGETVRISCKASGYTFTNYGMNWVKQAPGKGLKWMGWINTYTGEPTYAADFKRRFTFSLETSASTAYLQISNLKNDDTATYFCAKYPHYYGSSHWYFDVWGAGTTVTVSS

SEQ ID NO: 10 - protein, 108 aa, Mus musculus
DIQMTQTTSSLSASLGDRVIISCSASQDISNYLNWYQQKPDGTVKVLIYFTSSLHSGVPSRFSGSGSGTDYSLTISNLEPEDIATYYCQQYSTVPWTFGGGTKLEIKR

SEQ ID NO: 11 - protein, 113 aa, Artificial Sequence
  Synthesized
EVQLVESGGGLVQPGGSLRLSCAASGFTFSSYAMSWVRQAPGKGLEWVSVISGDGGSTYYADSVKGRFTISRDNSKNTLYLQMNSLRAEDTAVYYCARGFDYWGQGTLVTVSS

SEQ ID NO: 12 - protein, 108 aa, Artificial Sequence
  Synthesized
DIQMTQSPSSLSASVGDRVTITCRASQSISNYLAWYQQKPGKAPKLLIYAASSLESGVPSRFSGSGSGTDFTLTISSLQPEDFATYYCQQYNSLPWTFGQGTKVEIKR

SEQ ID NO: 13 - protein, 214 aa, Artificial Sequence
  Synthesized
DIQMTQSPSSLSASVGDRVTITCKASQSVSSDVGWYQQKPGKAPKLLIYSGSNRYSGVPSRFSGSGSGTDFTLTISSLQPEDFATYYCQQDYYSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 451 aa, Artificial Sequence
  Synthesized
EVQLVQSGAEVKKPGSSVKVSCKASGYTFTNYVINWVRQAPGQGLEWIGYNDPYNDVSKYNEKFKGRATITSDKSTSTAYMELSSLRSEDTAVYYCAKEGGGKYVYAMDSWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 212 aa, Artificial Sequence
  Synthesized
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 16 - protein, 457 aa, Artificial Sequence
  Synthesized
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

(19) United States
(12) Patent Application Publication
(10) Pub. No.: US 2015/0110788 A1
(43) Pub. Date: Apr. 23, 2015

(54) BISPECIFIC ANTIBODIES WITH AN FGF2 BINDING DOMAIN

(71) Applicant: GALAXY BIOTECH, LLC, Cupertino, CA (US)

(72) Inventors: Kyung Jin Kim, Cupertino, CA (US); Hangil Park, San Francisco, CA (US)

(73) Assignee: GALAXY BIOTECH, LLC, Cupertino, CA (US)

(21) Appl. No.: 14/382,544

(22) PCT Filed: Mar. 6, 2013
(86) PCT No.: PCT/US13/29415

§ 371 Date: Sep. 2, 2014

Related U.S. Application Data
(60) Provisional application No. 61/607,560, filed on Mar. 6, 2012.

(51) Int. Cl. C07K 16/22

(52) U.S. Cl. CPC C07K 16/22; C07K 2317/24; C07K 2317/21; C07K 2317/31; C07K 2317/56; C07K 2317/55; A61K 2039/505
USPC 424/136.1; 530/387.3; 435/328

(57) ABSTRACT

The present invention provides a bispecific antibody having a binding domain that binds to FGF2, a pharmaceutical composition comprising same, and methods of treatment comprising administering such a pharmaceutical composition to a patient.

### CROSS-REFERENCE TO RELATED APPLICATION

The present application is a nonprovisional and claims the benefit of 61/607,560 filed Mar. 6, 2012, incorporated by reference in its entirety for all purposes.

### FIELD OF THE INVENTION

The present invention relates generally to the combination of monoclonal antibody (mAb) and recombinant DNA technologies for developing novel biologics, and more particularly, for example, to the production of bispecific antibodies.

### BACKGROUND OF THE INVENTION

The Fibroblast Growth Factor (FGF) family plays important roles in embryonic development, tissue repair, angiogenesis and the growth of certain tumors (Ornitz et al., Genome Biol 2:REVIEWS3005, 2001; Presta et al., Cytokine Growth Factor Rev 16:159-78, 2005). The FGF family has 22 known members in humans, including FGF2 (also called basic FGF). Human FGF2 is an 18 kDa non-glycosylated polypeptide consisting of 146 amino acids in the mature form derived from a 155 aa precursor (Okada-Ban et al., 32:263-7, 2000).

FGF2 stimulates proliferation of fibroblasts and is involved in tissue remodeling and regeneration (Okada-Ban et al., op. cit.). FGF2 also induces migration, proliferation and differentiation of endothelial cells (Dow et al., Urology 55:800-06, 2000) so is a potent angiogenic factor (Presta et al., op. cit.). FGF2 is believed to play a role in cancer, both by stimulating angiogenesis and tumor cells directly (Presta et al., op. cit). FGF2 is strongly expressed in most gliomas (Takahashi et al., Proc Natl Acad Sci USA 87:5710-14, 1990), contributes to progression of prostate tumors (Dow et al., op. cit.), and is a key factor for the growth of melanomas (Wang et al., Nat Med 3:887-93, 1997). Overexpression of FGF2 and/or correlation with clinical features or outcome has also been reported for pancreatic cancer (Yamanaka et al., Cancer Res 53:5289-96, 1993), and other types of cancer.

The role of FGF2 in hepatocellular carcinoma (HCC; hepatoma) has been extensively studied and recently reviewed (Finn, Clin Cancer Res 16:390-7, 2010). Hepatomas are characterized by neovascularization, and angiogenesis plays a pivotal role in their growth, with FGF2 being an important pro-angiogenic factor (Ribatti et al., 32:437-44, 2006). FGF2 is overexpressed in HCC (Kin et al., J Hepatol 27:677-87, 1997) and higher serum level of FGF2 is an independent predictor of poor clinical outcome in HCC patients (Poon et al., Am J Surg 182:298-304, 2001). An anti-FGF2 mAb inhibited proliferation of many HCC cell lines, and administering the anti-FGF2 mAb locally at the site of the tumor inhibited growth of KIM-1 HCC xenografts (Ogasawara et al., Hepatology 24:198-205, 1996).

A number of antibodies that bind to and in some cases neutralize FGF2 have been described including the anti-FGF2 mAb 3H3, which was reported to suppress growth of U87MG and T98G glioma and HeLa cell xenografts (Takahashi et al., FEBS Let. 288:65, 1991) and growth of the K1000 FGF2-transfected 3T3 cell line in mice (Hori et al., Cancer Res. 51:6180, 1991). The anti-FGF2 mAb GAL-F2 and its humanized form HuGAL-F2 have been described in U.S. Pat. No. 8,101,725, which is incorporated herein by reference for all purposes; GAL-F2 inhibits the growth in mice of human RPMI 4788 colon tumor xenografts, and of human Hep-G2 and SMMC-7721 hepatocellular carcinoma xenografts.

“Cross-talk” between FGF2 and vascular endothelial growth factor (VEGF) has been reported, as has roles of these growth factors in angiogenesis and tumor growth (reviewed in Presta et al., op. cit. and Finn, op. cit.; Yoshiji et al., Hepatology 35:834-42, 2002). Upregulation of FGF2 expression has been reported in subjects resistant to Avastin® and other anti-VEGF drugs (Dempke et al., Eur J Cancer 45:1117-28, 2009; Casanovas et al., Cancer Cell 8:299-309, 2005; Bergers et al., Nat Rev Cancer 8:592-603, 2008).

### BRIEF SUMMARY OF THE INVENTION

In one embodiment, the invention provides a bispecific antibody with a first binding domain that binds human basic fibroblast growth factor (FGF2) and a second binding domain that binds another growth factor or receptor, for example VEGF. In a preferred embodiment, the first binding domain neutralizes FGF2. In particularly preferred embodiments, the first binding domain is the variable domain of the HuGAL-F2 mAb and/or the second binding domain is the variable domain of the humanized anti-VEGF mAb bevacizumab (Avastin®), A preferred antibody of the invention inhibits growth of a human tumor xenograft in a mouse, more preferably to a greater extent than antibodies containing only its first binding domain or second binding domain. Preferably, each part of the mAb of the invention is genetically engineered, e.g., chimeric, humanized or human. Cell lines producing such antibodies are also provided. In another embodiment, a pharmaceutical composition comprising a bispecific antibody of the invention is provided. In a third embodiment, the pharmaceutical composition is administered to a patient to treat cancer or other disease.

### BRIEF DESCRIPTION OF THE DRAWINGS

FIGS. 1A and B Inhibition of growth of HEP-G2 (A) and SMMC-7721 (B) human hepatocellular carcinoma xenografts by the indicated agents compared to negative control mAb hIgG. In the legends, “Both” indicates that HuGAL-F2 and Avastin® were both administered. The means of groups of 5-7 mice are shown; the error bars are S.E.M. HuGAL-F2 and Avastin® were administered i.p. at 5 mg/kg twice per week. The p values shown alongside the panels are the statistical significance between the indicated data points by Student's t test.

FIG. 2. SDS-PAGE of purified HuGAL-F2, Avastin®, and X-Ava/F2 under reducing conditions. The bands of X-Ava/F2 are identified by comparison to HuGAL-F2.

FIGS. 3A, B. Sequences of the (mature) HuGAL-F2 light chain (A) (SEQ ID NO:13) and HuGAL-F2 heavy chain (B) (SEQ ID NO:14) contained in X-Ava/F2. Sequential numbering is used. The first amino acid of the constant region is underlined, and in the heavy chain the amino acid that is substituted to create a knob is double underlined.

FIGS. 4A, B. Sequences of the (mature) Avastin® light chain (A) (SEQ ID NO:15) and Avastin® heavy chain (B) (SEQ ID NO:16), with C_{L} and C_{H}1 domains crossed over, contained in X-Ava/F2. Sequential numbering is used. The first amino acid of the constant region is underlined, and in the heavy chain the amino acids that are substituted to create a hole are double underlined.

FIG. 5. ELISA assay detecting antibody that binds both FGF2 and VEGF, applied to negative control human mAb hIgG, HuGAL-F2, Avastin® and X-Ava/F2.

FIGS. 6A, B. ELISA assay to compare binding of Avastin® and X-Ava/F2 to VEGF (A), and binding of HuGAL-F2 and X-Ava/F2 to FGF2 (B). The EC50 of each binding curve, as calculated by software, is shown.

FIG. 7. Inhibition of growth of HEP-G2 human hepatocellular carcinoma xenografts by the indicated agents compared to control PBS. The means of groups of 5-7 mice are shown. HuGAL-F2 and Avastin® were administered i.p. at 5 mg/kg twice per week, and X-Ava/F2 was administered i.p. twice per week at 5 mg/kg or 10 mg/kg as indicated. The curves other than for PBS are almost superimposed.

FIGS. 8A, B. Amino acid sequences of the HuGAL-F2 heavy chain (SEQ ID NO:5) and light chain mature variable regions (SEQ ID NO:2) are shown aligned with mouse GAL-F2 (SEQ ID NOS:4 and 1) and human acceptor V regions (SEQ ID NOS:6 and 3). The CDRs are underlined in the GAL-F2 sequences, and the amino acids substituted with mouse L2G7 amino acids are double underlined in the HuGAL-F2 sequences. The 1-letter amino acid code and Kabat numbering system are used for both the light and heavy chain.

FIGS. 9A shows sequences of the mature heavy chain variable regions of A.4.6.1, Avastin® (labeled Fab-12), and the heavy chain human acceptor used in humanizing A.4.6.1 to generate Avastin®, designated SEQ ID NOS. 9, 7 and 11 respectively. FIG. 9B shows sequences of the mature light chain variable regions of A.4.6.1, Avastin® (labeled Fab-12), and the light chain human acceptor used in humanizing A.6.1.1 to generate Avastin®, designated SEQ ID NOS. 10, 8 and 12 respectively. CDRs are underlined, and asterisks indicate amino acid differences.

### DETAILED DESCRIPTION OF THE INVENTION

More than one growth factor can contribute to growth of a tumor and that it may thus be necessary to block more than one growth factor to obtain an optimal therapeutic effect when treating cancer. The present application provides data showing that a combination of an anti-FGF2 antibody and an anti-VEGF antibody is more effective than either antibody alone in inhibiting tumor xenograft growth. Thus, the invention provides methods of inhibiting both FGF2 and VEGF or other growth factor. Simultaneous inhibition of the effects of FGF2 and another growth factor such as VEGF can be obtained with a bispecific antibody that has a binding domain for FGF2 and a separate binding domain for the other growth factor.

### 1 Antibodies

As used herein, “antibody” means a protein containing one or more domains capable of binding an antigen, where such domain(s) are derived from or homologous to the variable domain of a natural antibody. An “antigen” of an antibody means a compound to which the antibody specifically binds and is typically a polypeptide, but may also be a small peptide or small-molecule hapten or carbohydrate or other moiety. Examples of antibodies include natural, full-length tetrameric antibodies; antibody fragments such as Fv, Fab, Fab′ and (Fab′)₂; single-chain (scFv) antibodies (Huston et al., Proc. Natl. Acad. Sci. USA 85:5879, 1988; Bird et al., Science 242:423, 1988); single-arm antibodies (Nguyen et al., Cancer Gene Ther, 10:840, 2003); and bispecific, chimeric and humanized antibodies, as these terms are further explained below. Antibodies may be derived from any vertebrate species, including chickens, rodents (e.g., mice, rats and hamsters), rabbits, primates and humans. An antibody comprising a constant domain may be of any of the known isotypes IgG, IgA, IgM, IgD and IgE and their subtypes, i.e., human IgG1 IgG2 IgG3, IgG4 and mouse IgG1, IgG2a, IgG2b, and IgG3. The domains in an antibody can be identical to corresponding domains in a natural antibody or variants thereof as a result of, for example, humanization, (including veneering), chimerizing or affinity-maturing a natural antibody. The domains of an antibody (e.g., heavy and light chain variable regions preferably show at least 60%, 70%, 80%, 90% , 95%, 96, 97, 98, 99 or 100% identity to corresponding domains from a natural antibody.

A natural antibody molecule is generally a tetramer consisting of two identical heterodimers, each of which comprises one light chain paired with one heavy chain. Each light chain and heavy chain consists of a variable (V_{L} or V_{H}, or simply V) region followed by a constant (C_{L} or C_{H}, or simply C) region. The C_{H} region itself comprises C_{H}1, hinge (H), C_{H}2, and C_{H}3 regions. In 3-dimensional (3D) space, the V_{L} and V_{H} regions fold up together to form a V domain, which is also known as a binding domain since it binds to the antigen. The C_{L} region folds up together with the C_{H}1 region, so that the light chain V_{L}-C_{L} and the V_{H}-C_{H}1 region of the heavy chain together form a part of the antibody known as a Fab: a naturally “Y-shaped” antibody thus contains two Fabs, one from each heterodimer, forming the arms of the Y. The C_{H}2 region of one heterodimer folds up together with the C_{H}2 region of the other heterodimer, as do the respective C_{H}3 regions, forming together the single Fc domain of the antibody (the base of the Y), which interacts with other components of the immune system.

Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3D space to form the actual antibody binding site which locks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework, which forms the environment for the CDRs. Chothia et al., J. Mol. Biol. 196:901, 1987, have defined the related concept of hypervariable regions or loops determined by structure.

As used herein, a “genetically engineered” mAb is one for which the genes have been constructed or put in an unnatural environment (e.g., human genes in a mouse or on a bacteriophage or a cell line transfected with the genes) with the help of recombinant DNA techniques, and would therefore, e.g., not encompass a mouse mAb made with conventional hybridoma technology.

A humanized antibody (or respectively humanized antibody light or heavy chain) is a genetically engineered antibody (or respectively antibody light or heavy chain) in which CDRs from a mouse (or other non-human species such as chicken, rat, hamster or rabbit) antibody are grafted onto a human antibody (or respectively human antibody light or heavy chain), so the humanized antibody retains the binding specificity of the mouse antibody. The non-human antibody (or chain) from which the CDRs are derived is known as the “donor” antibody (or chain), and the human antibody into which they are grafted is known as the “acceptor” antibody (or chain). The sequence of the acceptor antibody (or chain) can be, for example, a mature human antibody sequence, a consensus sequence of human antibody sequences or a germline sequence. Thus, a humanized antibody is an antibody having CDRs from a donor antibody and a variable region framework and constant regions from a human antibody. A humanized antibody typically has both a humanized heavy chain and a humanized light chain; the acceptor light and heavy chains may come from the same or different human antibodies.

In order to retain high binding affinity in a humanized antibody, at least one of two additional structural elements can be employed. See, U.S. Pat. Nos. 5,530,101 and 5,585,089, incorporated herein by reference, which provide detailed instructions for construction of humanized antibodies. In the first structural element, the framework of the heavy chain variable region of the humanized antibody is chosen to have maximal sequence identity (between 65% and 95%) with the framework of the heavy chain variable region of the donor antibody, by suitably selecting the acceptor antibody from among the many known human antibodies. In the second structural element, in constructing the humanized antibody, selected amino acids in the framework of the human acceptor antibody (outside the CDRs) are replaced with corresponding amino acids from the donor antibody, in accordance with specified rules. Specifically, the amino acids to be replaced in the framework are chosen on the basis of their ability to interact with the CDRs. For example, the replaced amino acids can be adjacent to a CDR in the donor antibody sequence or within 4-6 angstroms of a CDR in the humanized antibody as measured in 3-dimensional space.

Other approaches to design humanized antibodies may also be used to achieve the same result as the methods in U.S. Pat. Nos. 5,530,101 and 5,585,089 described above, for example, “superhumanization” (see Tan et al. J. Immunol. 169: 1119, 2002, and U.S. Pat. No. 6,881,557) or the method of Studnicak et al., Protein Eng. 7:805, 1994. Moreover, other approaches to produce genetically engineered, reduced-immunogenicity mAbs include “reshaping”, “hyperchimerization” and veneering/resurfacing, as described, e.g., in Vaswami et al., Annals of Allergy, Asthma and Immunology 81:105, 1998; Roguska et al. Protein Eng. 9:895, 1996; and U.S. Pat. Nos. 6,072,035 and 5,639,641.

A chimeric antibody (or respectively chimeric antibody light or heavy chain) is an antibody (or respectively antibody light or heavy chain) in which the variable region of a mouse (or other non-human species) antibody (or respectively antibody light or heavy chain) is combined with the constant region of a human antibody; their construction by means of genetic engineering is well-known. Such antibodies retain the binding specificity of the mouse antibody, while being about two-thirds human. The proportion of nonhuman sequence present in mouse, chimeric and humanized antibodies suggests that the immunogenicity of chimeric antibodies is intermediate between mouse and humanized antibodies. Other types of genetically engineered antibodies include human antibodies made using phage display methods (Dower et al., WO91/17271; McCafferty et al., WO92/001047; Winter, WO92/20791; and Winter, FEBS Lett. 23:92, 1998, each of which is incorporated herein by reference) or by using transgenic animals (Lonberg et al., WO93/12227; Kucherlapati WO91/10741, each of which is incorporated herein by reference).

The term “antibody” also encompasses bispecific antibodies. A “bispecific antibody” is an antibody that contains a first domain binding to a first antigen and a second (different) domain binding to a second antigen, where the first and second domains are derived from or homologous to variable domains of natural antibodies. The first antigen and second antigen may be the same antigen, in which case the first and second domains can bind to different epitopes on the antigen. The term bispecific antibody encompasses multispecific antibodies, which in addition to the first and second domains contain one or more other domains binding to antigens and derived from or homologous to variable domains of natural antibodies. The term bispecific antibody also encompasses an antibody containing a first binding domain derived from or homologous to a variable domain of a natural antibody, and a second binding domain derived from another type of protein, e.g., the extracellular domain of a receptor, e.g., a VEGF receptor.

Thus, an exemplary binding domain comprises a light chain variable region and a heavy chain variable region from an antibody that itself binds to the antigen to be bound by the binding domain. Such a light chain variable region typically includes three light chain CDRs (CDRs L1, L2 and L3) within a light chain variable region framework. Likewise, the heavy chain variable region typically includes three heavy chain CDRs (CDRs H1, H2 and H3) with a heavy chain variable region framework. Light and heavy chains within a binding domain can be part of the same chain, usually separated by a spacer, as in an Fv fragment, or on separate chains, as on a Fab, Fab′ or intact antibody. Some or all of the light chain or heavy chain constant regions may also be present in the binding domain. If present, the light chain constant region can be contiguous with the light chain variable region and the heavy chain constant region can be contiguous with the heavy chain variable region. Any subregion or subregions of the heavy chain constant region can be present (i.e., CHL hinge, CH2, and/or CH3). Alternatively, a binding region can include a heavy or light chain dAb or a VHH chain from camelids or the like. The format of the two (or more) binding domains of a bispecific antibody can be the same or different as each other. In other words, one binding domain can be in the form of an Fv fragment and the other binding domain in the form of a Fab, or Fab′ or full-length heavy light chain pair, or a receptor extracellular domain.

Bispecific antibodies have been produced in a variety of forms, for example IgG-single chain variable fragment (scFv), Fab-scFv, and scFv-scFv fusion proteins (Coloma et al., Nat Biotechnol 15:125-6, 1997; Lu et al., J Immunol Methods 267:213-26, 2002; Mallender, J Biol Chem 269:199-206, 1994), dual variable domain antibodies (DVD-Ig; Wu et al., Nat Biotechnol 25:1290-7, 2007), and diabodies (Holliger et al., Proc Natl Acad Sci USA 90:6444-8, 1993). Bispecific F(ab′)₂ antibody fragments have been produced by chemical coupling (Brennan et al., Science 229:81, 1985) or by using leucine zippers (Kostelny et al., J Immunol 148:1547-53, 1992). A more naturally shaped bispecific antibody, with each heavy chain-light chain pair having a different V region, can be made by chemically cross-linking the two heavy chain-light chain pairs produced separately (Karpovsky et al., J Exp Med 160:1686-701, 1984), but such a method may not produce a sufficiently uniform product for commercial pharmaceutical use.

Naturally shaped bispecific antibodies can also be produced by expressing both required heavy chains and light chains in a single cell, made by fusing two hybridoma cell lines (a “quadroma”; Milstein et al., Nature 305: 537-40) or by transfection. However, because of mispairing between the respective chains, up to 10 different antibody-like compounds are made by such a cell (see Schaefer et al., Proc Natl Acad Sci USA 108:11187-92, 2011) so that it may be time consuming to purify the one desired bispecific antibody out of this mixture. An improved method incorporates protein engineering to insert an amino acid “knob” into the CH3 domain of one of the two heavy chains and a corresponding “hole” into the CH3 domain of the other so that the different heavy chains can more readily form heterodimers than homodimers, thus reducing formation of a non-bispecific antibody in which both heavy chains are the same (Ridgway et al., Protein Eng 9:617-21, 1996; Atwell et al., J Mol Biol 270:26-35, 1997; and U.S. Pat. No. 7,695,936, each of which is incorporated herein by reference for all purposes). However, there are still four different pairings of the two light chains with the two heavy chains, of which only one combination is correct. Thus, in a further improvement the “knobs-into-holes” method can be combined with an exchange or “crossing over” of heavy chain and light chain domains within the antigen binding fragment (Fab) of one light chain-heavy chain pair, so that in principle only the correct light chain-heavy chain pairs can form, thus creating bispecific antibodies called “CrossMabs” (Schaefer et al., Proc Natl Acad Sci USA 108:11187-92, 2011; WO 2009/080251; WO 2009/080252; WO 2009/080253; each of which is incorporated herein by reference for all purposes). In accordance with previous usage in the art, knobs and holes refer to mutations relative to the corresponding amino acid(s) of natural immunoglobulin sequences (e.g., as provided in the Swiss Prot database) that allow a knob (i.e.,. protrusion) to couple with a hole (i.e., an indentation) thereby promoting association of immunoglobulin chains bearing the knob and hole.

One or both of the light chain-heavy chain heterodimers in the bispecific antibody is typically genetically engineered, e.g., chimeric, humanized or human. For example, both heterodimers may be humanized (e.g., comprising both a humanized light chain and humanized heavy chain), or both heterodimers may be human, or one may be humanized and the other human. In addition, replacements can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

The epitope of a mAb is the region of its antigen to which the mAb binds. Two antibodies bind to the same or overlapping epitope if each competes for binding with the other, i.e., competitively inhibits (blocks) binding of the other to the antigen. That is, a 1× or 5× excess of one antibody inhibits binding of the other by at least 50% or 75%, or a 5×, 10×, 20× or 100× excess of one antibody inhibits binding of the other by at least 75%, preferably 90% or even 99%, as measured in a competitive binding assay (see, e.g., Junghans et al., Cancer Res. 50:1495, 1990). Alternatively, two antibodies have the same epitope on an antigen if they bind the same region of the antigen, or if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

A monoclonal antibody (mAb) (or its binding domain) that binds to a growth factor or respectively a cellular receptor (a “target”) is said to neutralize the growth factor or receptor, or be neutralizing, if the binding partially or completely inhibits one or more biological activities of the growth factor or receptor (e.g., when the mAb is used as a single agent), for example respectively its ability to bind to its receptor(s) or ligand(s). For example, among the biological properties of FGF2 or VEGF that a neutralizing antibody may inhibit are the ability of FGF2 or VEGF to bind to one or more of its receptors, to stimulate proliferation of certain cells including endothelial cells and various human tumor cells; to stimulate differentiation and migration of cells such as endothelial cells, or to stimulate angiogenesis, for example as measured by stimulation of human vascular endothelial cell (HUVEC) proliferation or tube formation or by induction of blood vessels when applied to the chick embryo chorioallantoic membrane (CAM). For a bispecific antibody, preferably each binding domain neutralizes one or more biological activities of the growth factor or receptor to which it binds.

With respect to each biological function, including specifically each biological function mentioned above, preferably a neutralizing mAb (or its binding domain) at a concentration of, e.g., 0.01, 0.1, 0.5, 1, 2, 5, 10, 20 or 50 mg/ml inhibits a biological function of the growth factor or receptor to which it binds by about at least 50% but preferably 75%, more preferably by 90% or 95% or even 99%, and most preferably approximately 100% (essentially completely) as assayed by methods known in the art. For a growth factor, typically the extent of inhibition is measured when the amount of growth factor used is just sufficient to fully stimulate the biological activity, or is 1, 2, or 5 ng/ml or 0.01, 0.02, 0.05, 0.1, 0.5, 1, 3 or 10 μg/ml. Most preferably, the mAb neutralizes not just one but two, three or several of the biological activities listed above; for purposes herein, a mAb (or binding domain) that neutralizes all the biological activities of a growth factor or receptor is called “fully neutralizing”.

Once a single, archetypal anti-human-FGF2 mAb, for example GAL-F2, has been isolated that has the desired properties described herein of neutralizing FGF2, it is straightforward to generate other mAbs with similar properties by using art-known methods, including mAbs that compete with GAL-F2 for binding to FGF2 and/or have the same epitope. For example, mice may be immunized with FGF2, hybridomas produced, and the resulting mAbs screened for the ability to compete with the archetypal mAb for binding to FGF2. Mice can also be immunized with a smaller fragment of FGF2 containing the epitope to which GAL-F2 binds. The epitope can be localized by, e.g., screening for binding to a series of overlapping peptides spanning FGF2. Alternatively, the method of Jespers et al., Biotechnology 12:899, 1994, which is incorporated herein by reference, may be used to guide the selection of mAbs having the same epitope and therefore similar properties to the archetypal mAb, e.g., GAL-F2. Using phage display, first the heavy chain of the archetypal antibody is paired with a repertoire of (preferably human) light chains to select an FGF2-binding mAb, and then the new light chain is paired with a repertoire of (preferably human) heavy chains to select a (preferably human) FGF2-binding mAb having the same epitope as the archetypal mAb. Alternatively variants of GAL-F2 can be obtained by mutagenesis of cDNA encoding the heavy and light chains of GAL-F2 obtained from the hybridoma.

In one embodiment, the present invention provides a bispecific antibody with a first binding domain that binds human basic fibroblast growth factor (FGF2) and a second binding domain that binds another growth factor or receptor, for example VEGF (collectively, the “targets”). Preferably the first or second binding domain or both neutralize or fully neutralize their target as described above, e.g., the FGF2-binding domain neutralizes FGF2. Each binding domain of the bispecific antibody is preferably specific for its target, that is it does not bind, or only binds to a much lesser extent (e.g., at least 10-fold or 100-fold less), other proteins (e.g., growth factors) that are related to the target, for example for FGF2 the other FGFs, e.g., FGF1. Some binding domains in the antibodies of the invention bind both human and mouse forms of the target, e.g. human and mouse FGF2, while other binding domains are specific for the human form, e.g., human FGF2. An antibody of the invention typically has a binding affinity (Ka) for one or both of its targets of at least 10⁷ M⁻¹ but preferably 10⁸M⁻¹ or higher, and most preferably 10⁹ M⁻¹ or higher or even 10¹⁰ M⁻¹ or higher.

Preferably, the bispecific antibody of the invention inhibits growth of a human tumor xenograft in a mouse, more preferably to a greater extent than antibodies containing only its first binding domain or second binding domain, most preferably to a substantially greater extent, that is by at least 25% or 50% greater extent or to a statistically significant difference (e.g., p<0.05). In some cases, treatment with the bispecific antibody inhibit growth of the xenograft more than additively (i.e., synergistically) relative to mAbs containing its individual binding domains, that is, the extent of inhibition by the bispecific antibody is greater than the sum of the extents of inhibition by mAbs containing its individual binding domains. In other words, a dose of bispecific antibody by mass can inhibit to a significantly greater extent (p<0.05) than an equal dose by mass of its component antibodies in equal proportions by mass. In some cases, the bispecific antibody, but not mAbs containing its individual binding domains, inhibit growth of a human tumor xenograft substantially completely, i.e., by at least 75% but preferably by at least 90% or 95% extent. As used herein, the “extent” of inhibition is determined as the percent reduction in the mean volume or weight of the xenografts in test antibody treated mice compared to control-treated mice, measured at a suitable time point, for example the last time point of the experiment (e.g., as shown in the experimental examples below). The xenograft may be a xenograft of human hepatocellular carcinoma cells, for example HEP-G2 cells (ATCC HB-8065) or SMMC-7721 cells.

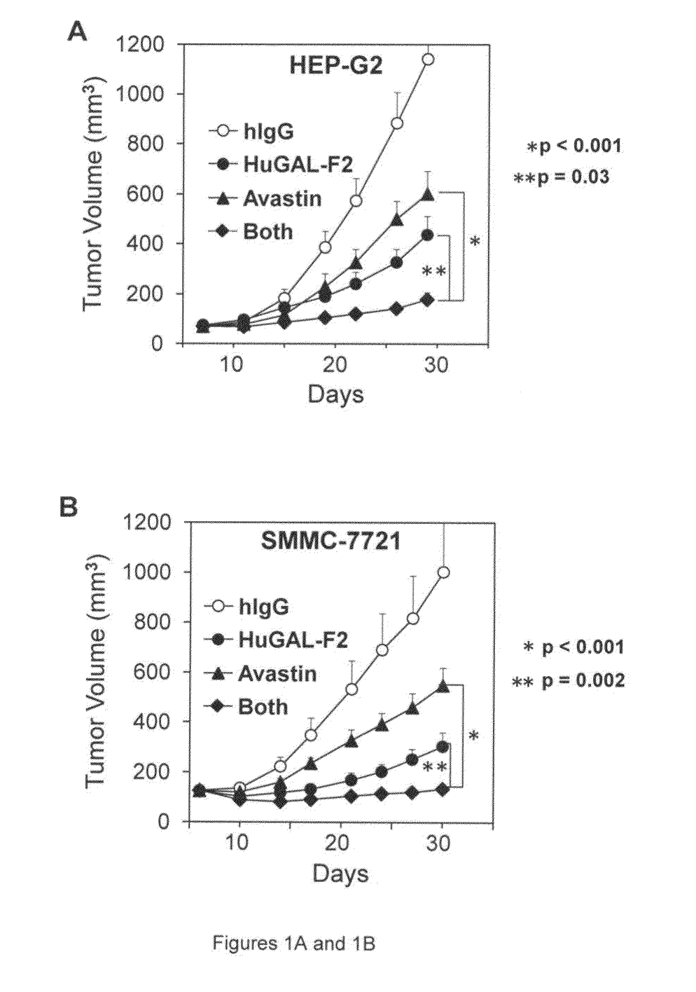

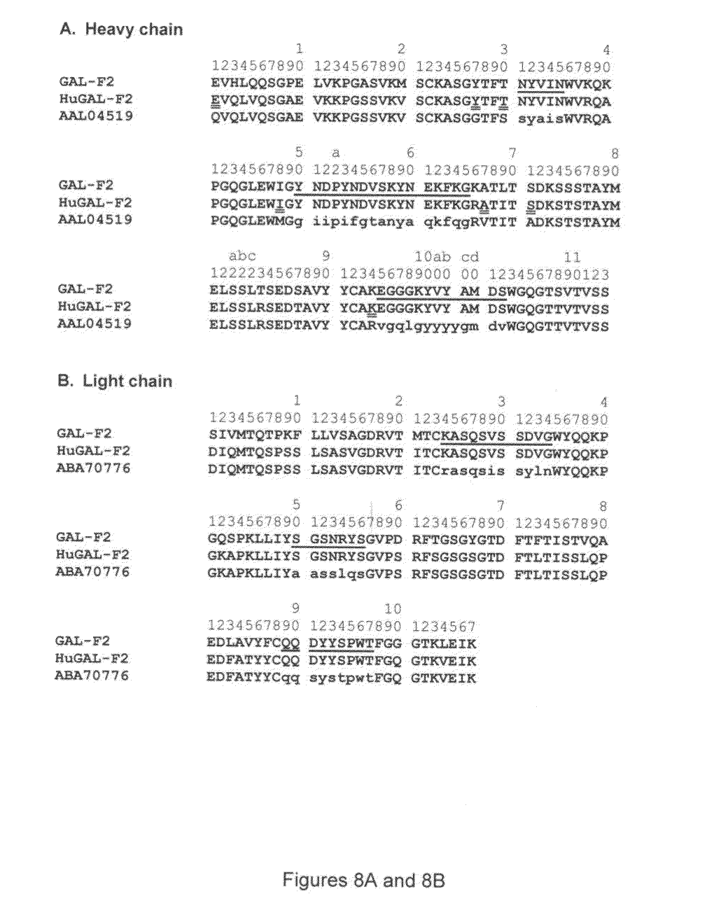

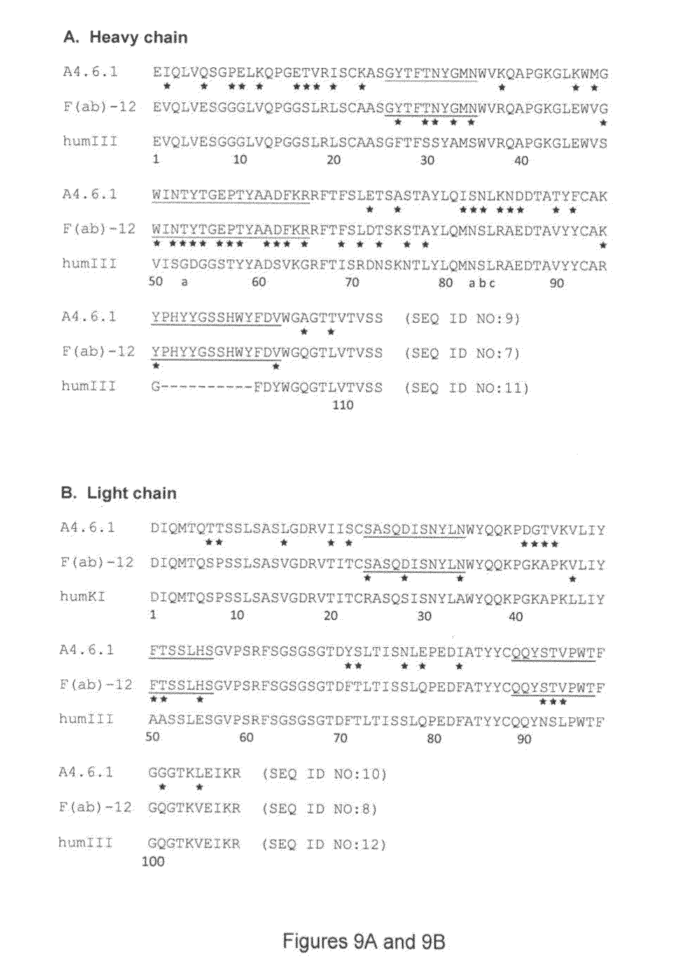

In a preferred embodiment of the invention, the first (FGF2-binding) domain of the bispecific antibody is the GAL-F2 (a mouse monoclonal antibody deposited as ATCC Number PTA-8864) variable domain or a humanized form of it, for example the HuGAL-F2 binding domain. In another preferred embodiment, the second binding domain is the variable domain of the bevacizumab mAb (Avastin®), which has been approved for the treatment of breast, colon and lung cancer and glioma (Avastin® label). Bevacizumab is a humanized form of the mouse antibody A4.6.1 (ATCC HB10709). Sequences of the heavy and light chain variable regions of bevacizumab are provided by FIG. 1 of U.S. Pat. No. 6,884,879 designated as F(ab)-12, incorporated by reference. Sequences of the light and heavy chain variable regions of GAL-F2, Hu-GAL-F2, A4.6.1 and bevacizumab (Fab-12) are reproduced in present FIGS. 8, 9A and 9B. Kabat CDRs are underlined except that for CDR H1 for the anti-VEGF sequences shown below the combined Kabat CDR-Chothia hypervariable region is underlined.

In another embodiment, the second binding domain is the variable domain of Lucentis® (ranibizumab), an affinity-optimized variant of bevacizumab (see Chen et al., J. Mol. Biol. 293, 865-881 (1999), incorporated by reference for all purposes, (see sequences of heavy and light chains designated Y0317 in FIG. 1). Additional antibodies include the G6 or B20 series antibodies (e.g., G6-31, B20-4.1), as described in WO2005/012359, WO2005/044853, and US2009-0142343 and other anti-VEGF antibodies described in any of U.S. Pat. Nos. 7,060,269, 6,582,959, 6,703,020; 6,054,297; WO98/45332; WO 96/30046; WO94/10202; EP 0666868B1; US2006009360, 20050186208, 20030206899, 20030190317, 20030203409, and 20050112126; and Popkov et al., Journal of Immunological Methods 288:149-164 (2004), each incorporated by reference for all purposes. Neutralizing mAbs with the same or overlapping epitope as GAL-F2 or bevacizumab, e.g., that compete for binding to the respective growth factors, provide other examples of variable domains that may be used as respectively the first and second binding domains in the bispecific antibody. Bispecific mAbs comprising variable domains that are at least 90%, 95% or 99% identical in amino acid sequence to those of GAL-F2 or HuGAL-F2 and/or bevacizumab and/or maintain their functional properties, and/ or which differ from them by a small number of functionally inconsequential amino acid substitutions (e.g., conservative substitutions), deletions, or insertions are also included in the invention. Bispecific antibodies having at least one and preferably all six CDR(s) that are at least 90%, 95% or 99% or 100% identical to the corresponding CDRs of GAL-F2 and/or bevacizumab are also encompassed. Bispecific antibodies having a first domain including at least one and preferably all 6 CDR(s) that are at least 90%, 95% or 99% or 100% identical to the corresponding CDRs of a monoclonal to FGF2 and a second domain including at least one and preferably all 6 CDR(s) that are at least 90%, 95% or 99% or 100% identical to the corresponding CDRs of a monoclonal to VEGF or other growth factor or receptor disclosed herein are also included. Here, as elsewhere in this application, percentage sequence identities are determined with antibody sequences maximally aligned by the Kabat numbering convention. After alignment, if a subject antibody region is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percent.

The term VEGF refers to any recognized member of the VEGF family, such as VEGF-A, VEGF-B, VEGF-C, VEGF-D or PIGF. Preferably, the second binding domain of the bispecific antibody binds to VEGF-A, and more preferably human VEGF-A, and such a bispecific antibody can be used in any of the embodiments described herein. An exemplary sequence for human VEGF-A is provided by Swiss-Prot P15692 of which the first 26 residues are a signal peptide removed in mature VEGF-A.

FGF2 preferably means human FGF2, for which a sequence is provided by, e.g., Ornitz et al., Genome Biol. 2: 3005.1, 2001 or Okada-Ban et al., Int. J. Biochem. Cell. Biol. 32:263, 2000, also Locus P09038 of Swiss-Prot database.

In other embodiments of the invention, the second binding domain of the bispecific antibody binds to another growth factor such as epidermal growth factor, any of the FGFs other than FGF2, hepatocyte growth factor (HGF), tumor necrosis factor (TNF), transforming growth factor beta (TGF-β1, TGF-β2, or TGF-β3), and any form of platelet derived growth factor (PDGF) or neuregulin or heregulin, or alternatively any extracellular domains of any receptor for these growth factors. Binding to human forms of these growth factors or receptor is preferred. Exemplary sequences are readily available from e.g., the Swiss-Prot database. The binding (variable) domain of the anti-HGF mAb HuL2G7 described in U.S. Pat. No. 7,632,926 (which is herein incorporated by reference for all purposes), or a binding domain comprising one or more of its CDRs, is especially preferred.

In preferred embodiments of the invention, correct pairing of the two light chain-heavy heterodimers of the bispecific antibody respectively comprising the first and second binding domains is promoted by inserting knobs and holes into the C_{H}3 regions of the respective heavy chains (Ridgway et al., Protein Eng 9:617-21, 1996; Atwell et al., J Mol Biol 270:26-35, 1997; and U.S. Pat. No. 7,695,936), while correct pairing of the light and heavy chains to form each heterodimer is promoted by “crossing over” of heavy chain and light chain domains within one of the heterodimers (Schaefer et al., Proc Natl Acad Sci USA 108:11187-92, 2011; WO 2009/080251; WO 2009/080252; WO 2009/080253). Thus, in a particularly preferred embodiment, the bispecific antibody comprises or consists of the following sequences with appropriate modifications described below: one heterodimer comprises or consist of a light chain-heavy chain pair of HuGAL-F2 (humanized GAL-F2), the sequence of which is provided in FIG. 13 of U.S. Pat. No. 8,101,725; while the other heterodimer consists of a light chain-heavy chain pair of the humanized anti-VEGF antibody having V regions with the sequence of F(ab)-12 in FIG. 1 of U.S. Pat. No. 6,884,879, (which is incorporated herein by reference for all purposes) respectively linked to a human kappa C region and human gamma-1 C region. (Sequences of these C regions are included in the provided sequences of HuGAL-F2). The appropriate sequence modifications mentioned above consist of the following: (1) creation of knobs and holes by the pair of substitutions T366Y in the HuGAL-F2 heavy chain and Y407T in the anti-VEGF mAb heavy chain, using Kabat numbering, and (2) domain crossing over by replacing the C_{L} domain of the anti-VEGF mAb light chain with the C_{H}1 domain of the anti-VEGF mAb heavy chain, and replacing the C_{H}1 domain of the anti-VEGF mAb heavy chain with the C_{L} domain of the anti-VEGF mAb light chain.

In addition to the bispecific antibody whose sequence is fully described in the paragraph above, alternative sequence modifications may be made. For example, to introduce knobs and holes, the pair of substitutions T366W in the HuGAL-F2 heavy chain and Y407A in the anti-VEGF mAb heavy chain may be made, or alternatively T366W in the HuGAL-F2 heavy chain may be combined with the three substitutions T366S, L368A and Y407V in the anti-VEGF mAb heavy chain. Or in each case described in the previous paragraph and this one, the substitutions in the HuGAL-F2 and anti-VEGF mAb heavy chains may be reversed. Regarding crossing over, the V_{L} and V_{H} domains of the anti-VEGF mAb may be switched (crossed over) instead of the C_{L} and C_{H}1 domains as described above, or the entire V_{L}-C_{L} region may be switched with the V_{H}-C_{H}1 region. And any of these cross overs may be done in the HuGAL-F2 heterodimer instead of in the anti-VEGF mAb heterodimer.

The invention provides also variant bispecific antibodies whose light and heavy chain differ from the ones specifically described above by a small number (e.g., typically no more than 1, 2, 3, 5 or 10) of replacements, deletions or insertions, usually in the C region or V region framework but possibly in the CDRs. Most often the replacements made in the variant sequences are conservative with respect to the replaced amino acids. Amino acids can be grouped as follows for determining conservative substitutions, i.e., substitutions within a group: Group I (hydrophobic sidechains): met, ala, val, leu, ile; Group II (neutral hydrophilic side chains): cys, ser, thr; Group III (acidic side chains): asp, glu; Group IV (basic side chains): asn, gln, his, lys, arg; Group V (residues influencing chain orientation): gly, pro; and Group VI (aromatic side chains): trp, tyr, phe. Substitutions can be made in the constant regions to reduce or increase effector function such as complement-mediated cytotoxicity or ADCC (see , e.g., U.S. Pat. No. 5,624,821, UA5,834,597, Lazar et al., PNAS 103, 4005 (2006) or to prolong half-life in humans (see, e.g., Hinton et al., J. Biol. Chem. 279, 6213 (2004). Exemplary substitutions include a Gln at position 250 and/or Leu at position 428 (EU numbering). Substitutions at any or all of positions 234, 235, 236 and/or 237 (EU numbering) reduce affinity for Fcy receptors (see, e.g., U.S. Pat. No. 6,624,821).

Preferably, replacements in the bispecific antibody have no substantial effect on the binding affinity or potency of the antibody, that is, on its ability to neutralize the biological activities of FGF2 and the target of the second binding domain. Preferably the variant sequences are at least 90%, more preferably at least 95%, and most preferably at least 98% identical to the original sequences. In addition, other allotypes or isotypes of the constant regions may be used.

Exemplary bispecific antibodies include bispecific antibodies comprising a first light chain having an amino acid sequence at least 90%, 95%, 96%, 97%, 98% or 99% identity to SEQ ID NO:13, a first heavy chain having an amino acid sequence at least 90%, 95%, 96%, 97%, 98% or 99% identity to SEQ ID NO:14, a second light chain having an amino acid sequence at least 90%, 95%, 96%, 97%, 98% or 99% identity to SEQ ID NO:15 and a second heavy chain having an amino acid sequence at least 90%, 95%, 96%, 97%, 98% or 99% identity to SEQ ID NO:16. In some bispecific antibodies, the first light chain comprises a HuGAL-F2 mature light chain variable region and human kappa light chain, the first heavy chain comprises a HuGAL-F2 mature heavy chain variable region, and CH1, CH2 and CH3 constant regions of human IgG1 isotype, the second light chain comprises a bevacizumab mature light chain variable region and a CH1 region of human IgG1 isotype; and the second heavy chain comprises a bevacizumab mature heavy chain, a human kappa light chain, and CH2 and CH3 constant regions of human IgG1 isotype. In some bispecific antibodies, the constant regions of the first heavy chain include one or more mutated residues relative to a natural human IgG1 sequence to form a knob, and the CH2 and CH3 constant regions of the second heavy chain include one or more mutated residues relative to a natural human IgG1 sequence to form a hole, wherein coupling of the knob and hole promotes association of the first and second heavy chains. In some bispecific antibodies, the first light chain has an amino acid sequence designated SEQ ID NO:13, the first heavy chain has an amino acid sequence designated SEQ ID NO:14, except the C-terminal lysine may be absent, the second light chain has an amino acid sequence designated SEQ ID NO:15, and the second heavy chain has an amino acid sequence designated SEQ ID NO:16 except the C-terminal lysine may be absent. Some of the above bispecific antibodies show greater inhibition of growth of a xenograft, e.g., a HEP-G2 xenograft, compared with an equal total dose by mass of HuGAL-F2 and bevacizumab (in equal proportions by mass).

The bispecific antibodies of the invention may be expressed by a variety of art-known methods. For example, genes encoding their light and heavy chain V regions may be synthesized from overlapping oligonucleotides and inserted together with available or synthesized C regions into expression vectors (e.g., commercially available from Invitrogen) that provide the necessary regulatory regions, e.g., promoters, enhancers, poly A sites, etc. Use of the CMV promoter-enhancer is preferred. The expression vectors may then be transfected using various well-known methods such as lipofection or electroporation into a variety of mammalian cell lines such as CHO or non-producing myelomas including Sp2/0 and NS0, and cells expressing the antibodies selected by appropriate antibiotic selection. See, e.g., U.S. Pat. No. 5,530,101. Larger amounts of antibody may be produced by growing the cells in commercially available bioreactors.

Once expressed, the bispecific antibodies of the invention may be purified according to standard procedures of the art for purifying mAbs, such as microfiltration, ultrafiltration, protein A or G affinity chromatography, size exclusion chromatography, anion exchange chromatography, cation exchange chromatography and/or other forms of affinity chromatography based on the targets for the bispecific antibodies or organic dyes or the like. Substantially pure antibodies of at least about 90 or 95% homogeneity are preferred, and 98% or 99% or more homogeneity most preferred, for pharmaceutical uses. It will also be understood that when the bispecific antibody is manufactured by conventional procedures, one to several amino acids at the amino or carboxy terminus of the light and/or heavy chain, such as the C-terminal lysine of the heavy chain, may be missing or derivatized in a proportion or all of the molecules, and such a composition will still be considered to be the bispecific antibody.

### 2. Treatment Methods

The invention provides methods of treatment in which the bispecific antibody of invention is administered to patients having a disease (therapeutic treatment) or at risk of occurrence or recurrence of a disease (prophylactic treatment). The term “patient” includes human patients; veterinary patients, such as cats, dogs and horses; farm animals, such as cattle, sheep, and pigs. The methods are particularly amenable to treatment of human patients. The FGF2 binding domain used in methods of treating human patients binds to the human FGF2 protein, the sequence of which is provided by, e.g., Ornitz et al, Genome Biol. 2: 3005.1, 2001 or Okada-Ban et al, int J. Biochem. Cell Biol. 32:263, 2000, also Locus P09038 of Swiss-Prot database. An antibody to a human protein can also be used in other species in which the species homolog has antigenic crossreactivity with the human protein. In species lacking such crossreactivity, an antibody is used with appropriate specificity for the species homolog present in that species. However, in xenograft experiments in laboratory animals, an antibody with specificity for the human protein expressed by the xenograft is generally used.

In a preferred embodiment, the present invention provides a pharmaceutical formulation comprising the antibodies described herein. Pharmaceutical formulations of he antibodies contain the antibody in a physiologically acceptable carrier, optionally with excipients or stabilizers, in the form of lyophilized or aqueous solutions. Acceptable carriers, excipients or stabilizers are nontoxic to recipients at the dosages and concentrations employed, and include buffers such as phosphate, citrate, or acetate at a pH typically of 5.0 to 8.0, most often 6.0 to 7.0; salts such as sodium chloride, potassium chloride, etc. to make isotonic; antioxidants, preservatives, low molecular weight polypeptides, proteins, hydrophilic polymers such as polysorbate 80, amino acids, carbohydrates, chelating agents, sugars, and other standard ingredients known to those skilled in the art (Remington's Pharmaceutical Science 16th edition, Osol, A. Ed. 1980). The antibody is typically present at a concentration of 1-100 mg/ml, e.g., 10 mg/ml.

In another preferred embodiment, the invention provides a method of treating a patient with a disease using a bispecific antibody of the invention (i.e., containing an FGF2-binding domain) in a pharmaceutical formulation. The antibody prepared in a pharmaceutical formulation can be administered to a patient by any suitable route, especially parentally by intravenous infusion or bolus injection, intramuscularly or subcutaneously. Intravenous infusion can be given over as little as 15 minutes, but more often for 30 minutes, or over 1, 2 or even 3 hours. The antibody can also be injected directly into the site of disease (e.g., a tumor), or encapsulated into carrying agents such as liposomes. The dose given is sufficient to at least partially alleviate the condition being treated (“therapeutically effective dose”) and is optionally 0.1 to 5 mg/kg body weight, for example 1, 2, 3 or 4 mg/kg, but may be as high as 10 mg/kg or even 15, 20 or 30 mg/kg, e.g., 1 to 10 mg/kg, 1 to 20 mg/kg or 1 to 30 mg/kg. A fixed unit dose may also be given, for example, 100, 200, 500, 1000 or 2000 mg, or the dose may be based on the patient's surface area, e.g., 1000 mg/m². Usually between 1 and 8 doses, (e.g., 1, 2, 3, 4, 5, 6, 7 or 8) are administered to treat cancer, but 10, 20 or more doses may be given. The antibody can be administered daily, biweekly, weekly, every other week, monthly or at some other interval, depending, e.g. on the half-life of the antibody, for 1 week, 2 weeks, 4 weeks, 8 weeks, 3-6 months or longer or until the disease progresses. Repeated courses of treatment are also possible, as is chronic administration.

A combination of a dose, frequency of administration and route of administration effective to at least partially alleviate a disease present in a patient being treated is referred to as a therapeutically effective regime. A combination of a dose, frequency of administration and route of administration effective to inhibit or delay onset of a disease in a patient is referred to as a prophylactically effective regime.

Diseases especially susceptible to treatment with the antibodies of this invention include solid tumors believed to require angiogenesis, or to be associated with elevated levels of FGF2 and/or VEGF, or to be associated with expression of FGF2 and/or VEGF. Such tumors, for which treatment with the bispecific antibody is appropriate, include for example ovarian cancer, breast cancer, lung cancer (small cell or non-small cell), colon cancer, prostate cancer, cervical cancer, endometrial cancer, renal cell carcinoma, pancreatic cancer, gastric cancer, esophageal cancer, head-and-neck tumors, hepatocellular carcinoma or hepatoma (liver cancer), which is an especially preferred disease indication, melanoma, sarcomas, carcinomas, and brain tumors (e.g., gliomas such as glioblastomas). Hematologic malignancies such as leukemias and lymphomas and multiple myeloma can also be susceptible to such treatment. Other diseases associated with angiogenesis for which treatment with the antibodies of the invention are suitable include age-related macular degeneration (AMD), diabetic retinopathy, neovascular glaucoma and other diseases of the eye; psoriasis and other diseases of the skin; and rheumatoid arthritis.

In a preferred embodiment, the bispecific antibody is administered in combination with (i.e., together with, that is, before, during or after) other therapy. For example, to treat cancer, the antibody may be administered together with any one or more of the known chemotherapeutic drugs, for example alkylating agents such as carmustine, chlorambucil, cisplatin, carboplatin, oxaliplatin, procarbazine, and cyclophosphamide; antimetabolites such as fluorouracil, floxuridine, fludarabine, gemcitabine, methotrexate and hydroxyurea; natural products including plant alkaloids and antibiotics such as bleomycin, doxorubicin, daunorubicin, idarubicin, etoposide, mitomycin, mitoxantrone, vinblastine, vincristine, and Taxol (paclitaxel) or related compounds such as Taxotere®; the topoisomerase 1 inhibitor irinotecan; agents specifically approved for brain tumors including temozolomide and Gliadel® wafer containing carmustine; and inhibitors of tyrosine kinases such as Gleevec® (imatinib mesylate), Sutent® (sunitinib malate), Nexavar® (sorafenib), Tarceva® (erlotinib) and Iressa® (gefitinib) and Zelboraf® (vemurafenib); inhibitors of angiogenesis; and all approved and experimental anti-cancer agents listed in WO 2005/017107 A2 (which is herein incorporated by reference). The antibody may be used in combination with 1, 2, 3 or more of these other agents used in a standard chemotherapeutic regimen. Normally, the other agents are those already known to be effective for the particular type of cancer being treated. The bispecific antibody is especially useful in overcoming resistance to chemotherapeutic drugs and thereby increasing their effectiveness (see Song et al. Proc. Natl. Acad. Sci USA 97:8658, 2000).

Other agents with which the bispecific antibodies of the invention can be administered to treat cancer include biologics such as monoclonal antibodies, including Herceptin® against the HER2 antigen; Avastin® against VEGF; or antibodies to the Epidermal Growth Factor (EGF) receptor such as Erbitux® (cetuximab) and Vectibix® (panitumumab). Antibodies against Hepatocyte Growth Factor (HGF) are especially preferred for use with the bispecific antibody, including mAb L2G7 (Kim et al., Clin Cancer Res 12:1292, 2006 and U.S. Pat. No. 7,220,410) and particularly its chimeric and humanized forms such as HuL2G7 (U.S. Pat. No. 7,632,926); the human anti-HGF mAbs described in WO 2005/017107 A2, particularly 2.12.1; and the HGF binding proteins described in WO 07143090 A2 or WO 07143098 A2; and other neutralizing anti-HGF mAbs that compete for binding with any of the aforementioned mAbs. A mAb that binds the cMet receptor of HGF is also preferred, for example the anti-cMet mAb OA-5D5 (Martens et al., Clin. Cancer Res. 12:6144, 2006; also designated “MetMab”) that has been genetically engineered to have only one “arm”, i.e. binding domain. Moreover, the bispecific antibody can be used together with any form of surgery and/or radiation therapy including external beam radiation, intensity modulated radiation therapy (IMRT) and any form of radiosurgery such as, e.g. Gamma Knife.

Treatment (e.g., standard chemotherapy) including the bispecific antibody may alleviate cancer by increasing the median progression-free survival or overall survival time of patients by at least 30% or 40% but preferably 50%, 60% to 70% or even 100% or longer, or by at least 2 or 3 or 6 months, compared to the same treatment (e.g., chemotherapy) but without the bispecific antibody. In addition or alternatively, treatment (e.g., standard chemotherapy) including the bispecific antibody may increase the complete response rate, partial response rate, or objective response rate (complete+partial) of patients with these tumors by at least 30% or 40% but preferably 50%, 60% to 70% or even 100% compared to the same treatment (e.g., chemotherapy) but without the antibody.

Typically, in a clinical trial (e.g., a phase II, phase II/III or phase III trial), the aforementioned increases in median progression-free survival and/or response rate of the patients treated with chemotherapy plus the bispecific antibody, relative to the control group of patients receiving chemotherapy alone (or plus placebo), are statistically significant, for example at the p=0.05 or 0.01 or even 0.001 level. The complete and partial response rates are determined by objective criteria commonly used in clinical trials for cancer, e.g., as listed or accepted by the National Cancer Institute and/or Food and Drug Administration. Similarly, the determination of whether a patient has progressed under treatment is typically made according to the RECIST (Response Evaluation Criteria In Solid Tumors) criteria.

### 3. Other Methods

The bispecific antibodies of the invention also find use in diagnostic, prognostic and laboratory methods. They may be used to measure the level of FGF2 in a tumor or in the circulation of a patient with a tumor, to determine if the level is measurable or even elevated, and therefore to follow and guide treatment of the tumor, since tumors associated with measurable or elevated levels of FGF2 are most susceptible to treatment with a bispecific antibody comprising an FGF2-binding domain. For example, a tumor associated with high levels of FGF2 and/or VEGF would be especially susceptible to treatment with such an antibody. In particular embodiments, the antibodies can be used in an ELISA or radioimmunoassay to measure the level of FGF2, e.g., in a tumor biopsy specimen or in serum or in media supernatant of FGF2-secreting cells in cell culture. For various assays, the antibody may be labeled with fluorescent molecules, spin-labeled molecules, enzymes or radioisotopes, and may be provided in the form of kit with all the necessary reagents to perform the assay. In other uses, the antibodies are used to purify FGF2, e.g., by affinity chromatography.

### EXAMPLES

### Example 1

Human hepatocellular carcinoma (HCC) cell lines were grown in complete DMEM medium and harvested in PBS. Female 5 to 6-week-old athymic nude mice were injected s.c. with 10⁷ SMMC-7721 cells or 2×10⁶ HEP-G2 cells in 0.1 ml PBS in the dorsal area. When the tumor sizes reached ˜100 mm³, mice were grouped randomly (n=5-7/group) and the indicated antibodies (100 mg in 0.1 ml, equivalent to 5 mg/kg body weight) was administered i.p. twice per week. hIgG is negative control human antibody, and Both indicates that both HuGAL-F2 and Avastin® were administered. Tumor volumes were determined twice weekly by measuring in two dimensions, length (a) and width (b), and calculating volume as V=ab²/2. Statistical analysis was performed by Student's t test applied to the final time point.

Both HuGAL-F2 and Avastin® strongly inhibited growth of SMMC-7721 and HEP G2 xenografts, with HuGAL-F2 slightly more effective than Avastin®. Importantly, the combination of antibodies was more effective than either antibody alone, to a high level of statistical significance, as seen in FIGS. 1A and B, and indeed almost completely inhibited growth of the xenografts. This shows that a bispecific antibody having the binding domains of HuGAL-F2 and Avastin® is a more effective treatment than either the HuGAL-F2 or Avastin® antibodies and may inhibit growth of the xenograft substantially completely.

### Example 2

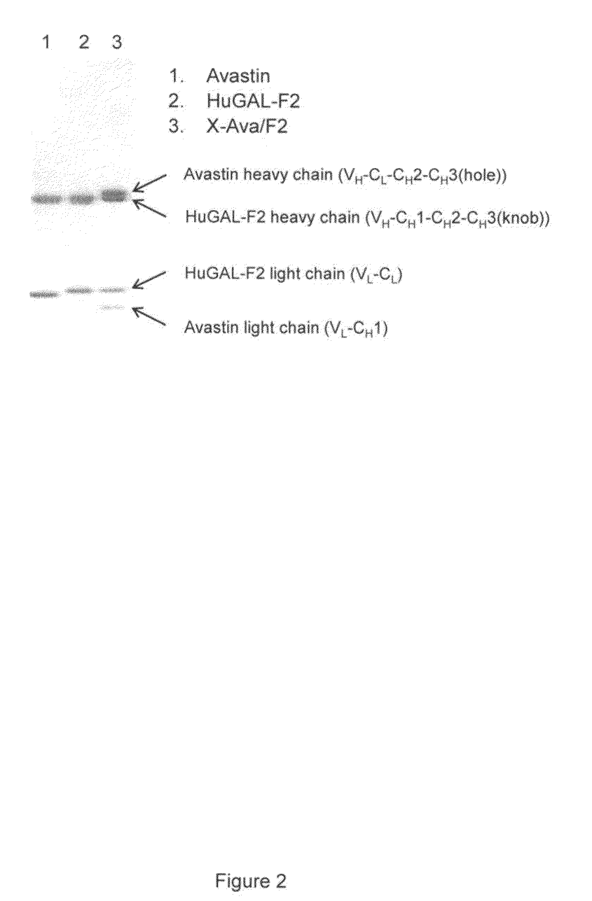

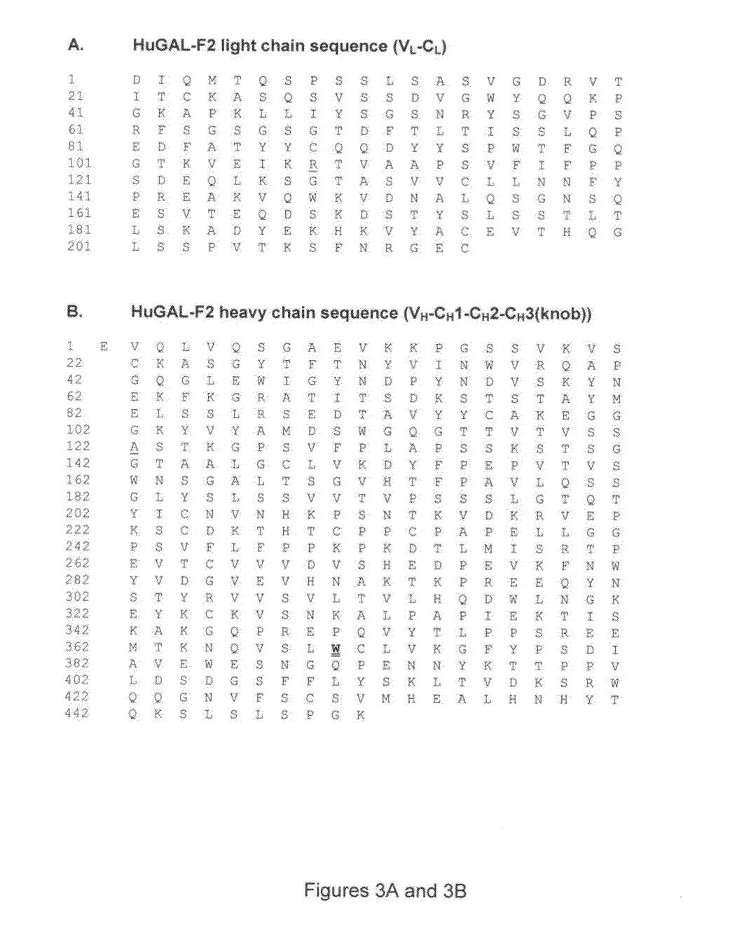

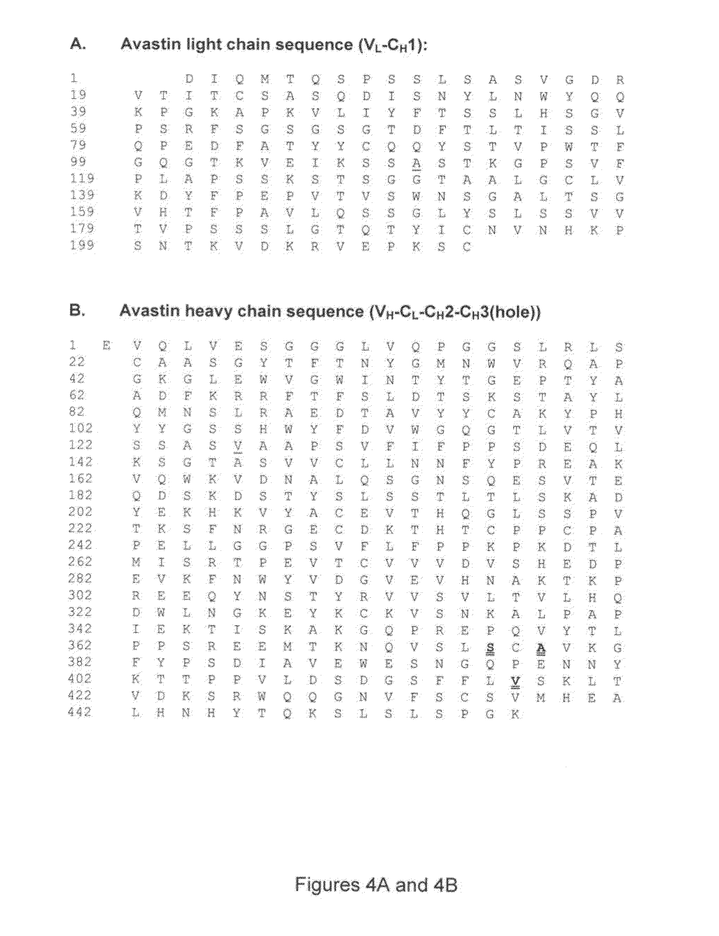

A bispecific antibody designated X-Ava/F2 was constructed as described above, using the domain crossing-over method, which comprises the HuGal-F2, variable domain sequence shown in FIGS. 8A, B and the Avastin® variable domain sequence (Fab-12) shown in FIGS. 9A, B. For this purpose, four genes encoding the desired protein sequences were synthesized using standard recombinant DNA methods: (1) HuGAL-F2 light chain as shown in FIG. 3A, (2) HuGAL-F2 heavy chain sequence with a knob at position 370 (in the sequential numbering of FIG. 3B) created by substituting Trp (W), as shown in FIG. 3B, (3) Avastin® light chain variable domain attached to the C_{H}1 heavy chain constant domain via a Ser-Ser (—S—S—) amino acid linker, as shown in FIG. 4A, (4) Avastin® heavy chain variable domain attached to the C_{L} domain via an Ala-Ser (-A-S—) linker, followed by the C_{H}2 and C_{H}3 domains, with a hole created in C_{H}3 by substitutions of Ser at position 376, Ala at position 378 and Val at position 417 (all in the sequential numbering of FIG. 4B), as shown in FIG. 4B. The four genes, which also encoded signal sequences preceding the mature protein sequences, were inserted into expression vectors, which were cotransfected together into mammalian cells for expression. The secreted antibody was purified from culture media using protein A chromatography as usual. Reduced SDS-PAGE of the purified mAb showed two heavy chain bands and two light chains (FIG. 2), one of each matching the respective HuGAL-F2 bands, and one of each having slightly different mobilities due to the domain swapping in the Avastin® chains.

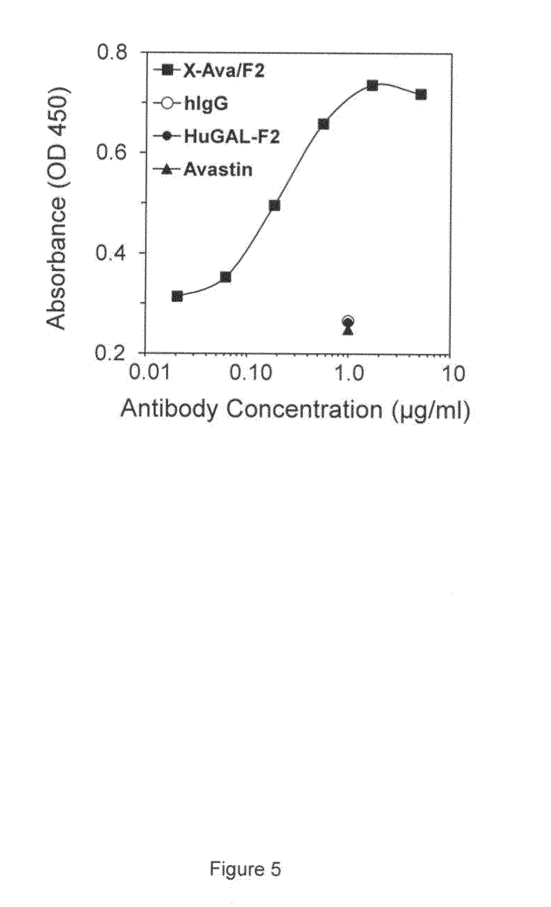

To verify that the purified antibody consisted primarily of X-Ava/F2 able to bind both FGF2 (via the HuGAL-F2 binding domain) and VEGF (via the Avastin® binding domain), an ELISA assay was used. Heparin (50 μg/ml) was bound to an ELISA plate overnight and used to capture VEGF (0.2 μg/ml) overnight, followed by 2% BSA for blocking. Wells of the plate were then incubated with increasing concentrations of X-Ava/F2 or fixed, high concentrations (1 μg/ml) of negative control human mAb hIgG, HuGAL-F2 or Avastin, followed by Flag-FGF2 (1 μg/ml; Flag peptide linked to FGF2), and then HRP-anti-Flag M2 antibody (Sigma Aldrich) and substrate for detection. In principle, this assay should only detect bispecific antibody, because any detectable antibody must bind both to the VEGF on the plate in order to be captured and to Flag-FGF2 in solution in order to be detected. Indeed, neither of the constituent mAbs HuGAL-F2 and Avastin® gave a signal above the negative control mAb hIgG in the assay (FIG. 5). In contrast, the purified X-Ava/F2 mAb produced a strong concentration-dependent signal, showing that the mAb binds both FGF2 and VEGF.

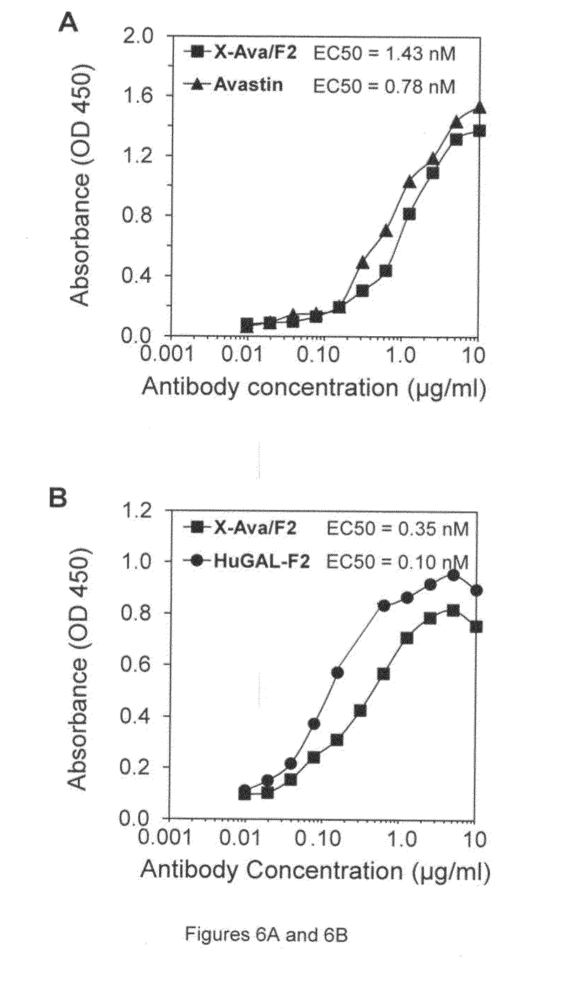

To more precisely compare the binding affinity of X-Ava/F2 for VEGF and FGF2 with that of Avastin® and HuGAL-F2 respectively, another ELISA assay was used. Heparin (50 μg/ml) was bound to ELISA plates overnight and used to capture either VEGF or FGF2 (0.2 μg/ml) overnight, followed by 2% BSA for blocking. Wells of the VEGF (respectively FGF2) plate were then incubated with increasing concentrations of X-Ava/F2 or Avastin® (resp. X-Ava/F2 or HuGAL-F2), followed by HRP-goat-anti-human-IgG-Fc and substrate for detection. The EC50 (antibody concentration for half-maximal binding) of X-Ava/F2 and Avastin® were within 2-fold (FIG. 6A), and the EC50 of X-Ava/F2 and HuGAL-F2 were within 4-fold (FIG. 6B). However, correcting for the fact that a mole of X-Ava/F2 only contains half as many binding sites as a mole of Avastin® or HuGAL-F2 (because each molecule only contains 1 binding site instead of 2 for each ligand), the binding affinity of X-Ava/F2 and Avastin® for VEGF are essentially the same, and the binding affinity of X-Ava/F2 and HuGAL-F2 for FGF2 are within 2-fold.

### Example 3

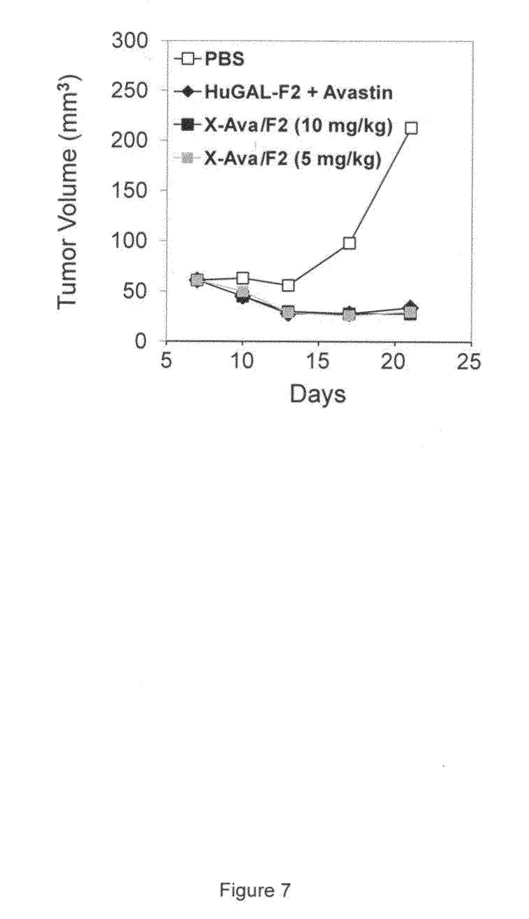

To determine the ability of X-Ava/F2 to inhibit growth of xenografts, an experiment with HEP-G-2 xenogafts was conducted in the same manner as in Example 1 above. Treatment with both, i.e., the combination of, HuGAL-F2 and Avastin® (5 mg/kg each twice/week) strongly inhibited growth of the xenografts (FIG. 7), just as in Example 1 (FIG. 1A). Moreover, either 5 mg/kg or 10 mg/kg X-Ava/F2 (twice per week) was as effective as treatment with both HuGAL-F2 and Avastin® in strongly (substantially completely) inhibiting growth of the xenografts (FIG. 7), In terms of total mass and the number of binding sites for each ligand, 10 mg/kg X-Ava/F2 is equivalent to 5 mg/kg each of HuGAL-F2 and Avastin, while 5 mg/kg X-Ava/F2 is only half as much, so the ability of this dose to inhibit as effectively as 5 mg/kg of each mAb indicates that the X-Ava/F2 bispecific antibody as a single agent is unexpectedly more effective than the combination of HuGAL-F2 and Avastin® and much more effective than each of these mAbs alone (by comparison with FIG. 1A).

Although the invention has been described with reference to presently preferred embodiments, it should be understood that various modifications can be made without departing from the invention. Unless otherwise apparent from the context any step, element, embodiment, feature or aspect of the invention can be used with any other. All publications, patents and patent applications including accession numbers and the like cited are herein incorporated by reference in their entirety for purposes to the same extent as if each individual publication, patent and patent application was specifically and individually indicated to be incorporated by reference in its entirety for all purposes. To the extent a citation such as an accession number is associated with different versions at different times, the version in effect at the effective filing date of the application is meant, the effective filing date being the actual filing date or earlier filing date of an application providing the relevant citation from which priority is claimed.

What is claimed is:

1. A bispecific antibody comprising a first binding domain that binds to human FGF2 and a second binding domain that binds a growth factor or growth factor receptor.

2. The bispec c antibody of claim 1 wherein the second binding domain binds to human VEGF.

3. The bispecific antibody of claim 1 wherein the first and second binding domains are each humanized or human.

4. The bispecific antibody of claim 3 wherein the first binding domain is the variable domain of the HuGAL-F2 antibody and the second binding domain is the binding domain of the bevacizumab antibody.

5. The bispecific antibody of claim 1 that inhibits growth of a human tumor xenograft in a mouse.

6. The bispecific antibody of claim 1, wherein the first binding domain comprises a light chain variable region and a heavy chain variable region of an antibody that binds to FGF2 and the second binding domain comprises a light chain variable region and a heavy chain variable region of an antibody that binds the growth factor or growth factor receptor.

7. The bispecific antibody of claim 6, wherein the first binding domain is an Fv, Fab, or Fab′ fragment and the second binding domain is an Fv, Fab, or Fab′ fragment,

8. The bispecific antibody of claim 1, wherein either or both of the first or second binding domain comprises a light chain variable region linked to a light chain constant region and a heavy chain variable region linked to a heavy chain constant region.

9. The bispecific antibody of any claim 2, wherein the second binding domain binds to human VEGF-A.

10. A cell line producing the bispecific antibody of claim 1.

11. A composition comprising a bispecific antibody of claim 1 in a pharmaceutically acceptable carrier.

12. A method of treating a disease in a patient by administering an effective regime of the pharmaceutical composition of claim 11 to a subject having or at risk of the disease.

13. The method of claim 12 where the disease is cancer.

14. The method of claim 13 wherein the cancer is hepatocellular carcinoma.

15. A bispecific antibody comprising a first binding domain that binds to FGF2 and a second binding domain that binds to VEGF, wherein the first binding domain comprises a light chain comprising the three CDRs of the light chain of HuGAL-F2 and a heavy chain comprising the three CDRs of the heavy chain of HuGAL-F2, and the second binding domain comprises a light chain comprising the three CDRs of the light chain of Avastin® and a heavy chain comprising the three CDRS of the heavy chain of Avastin.

16. A bispecific antibody comprising a first light chain having an amino acid sequence at least 95% identical to SEQ ID NO:13, a first heavy chain having an amino acid sequence at least 95% identical to SEQ ID NO:14, a second light chain having an amino acid sequence at least 95% identical to SEQ ID NO:15 and a second heavy chain having an amino acid sequence at least 95% identical to SEQ ID NO:16.

17. The bispecific antibody of claim 16, wherein the first light chain comprises a HuGAL-F2 mature light chain variable region and human kappa light chain, the first heavy chain comprises a HuGAL-F2 mature heavy chain variable region, and CH1, CH2 and CH3 constant regions of human IgG1 isotype, the second light chain comprises a bevacizumab mature light chain variable region and a CH1 region of human IgG1 isotype; and the second heavy chain comprises a bevacizumab mature heavy chain, a human kappa light chain, and CH2 and CH3 constant regions of human IgG1 isotype.

18. The bispecific antibody of claim 17, wherein the constant regions of the first heavy chain include one or more mutated residues relative to a natural human IgG1 sequence to form a knob, and the CH2 and CH3 constant regions of the second heavy chain include one or more mutated residues relative to a natural human IgG1 sequence to form a hole, wherein coupling of the knob and hole promotes association of the first and second heavy chains.

19. The bispecific antibody of claim 16, wherein the first light chain has an amino acid sequence designated SEQ ID NO:13, the first heavy chain has an amino acid sequence designated SEQ ID NO:14, except the C-terminal lysine may be absent, the second light chain has an amino acid sequence designated SEQ ID NO:15, and the second heavy chain has an amino acid sequence designated SEQ ID NO:16 except the C-terminal lysine may be absent.

20. The bispecific antibody of any of claims 16-19, which shows greater inhibition of growth of a HEP-G2 xenograft compared with an equal dose by mass of HuGAL-F2 and bevacizumab in equal proportions by mass.

21. A bispecific antibody comprising a first binding domain and a second binding domain, wherein the first binding domain comprises a light chain having the sequence of FIG. 3A and a heavy chain having the sequence of FIG. 3B, and the second binding domain comprises a light chain having the sequence of FIG. 4A and a heavy chain having the sequence of FIG. 4B.

22. A cell line producing a bispecific antibody of any of claims 16-21.

23. A pharmaceutical composition comprising a bispecific antibody of any of claims 16-21.

24. A method of treating a disease in a patient by administering the pharmaceutical composition of claim 23 to the patient.

25. The method of claim 24, wherein the disease is cancer.
